(19) United States
(12) Patent Application Publication
(10) Pub. No.: US 2022/0127685 A1
(43) Pub. Date: Apr. 28, 2022

(54) NUCLEIC ACID DETECTION SYSTEM AND NUCLEIC ACID DETECTION METHOD

(71) Applicant: CANON MEDICAL SYSTEMS CORPORATION, Otawara-shi (JP)

(72) Inventors: Chang TAN, Beijing (CN); Hao HUANG, Beijing (CN); Qiqi XU, Beijing (CN)

(73) Assignee: CANON MEDICAL SYSTEMS CORPORATION, Otawara-shi (JP)

(21) Appl. No.: 17/506,804

(22) Filed: Oct. 21, 2021

(30) Foreign Application Priority Data
Oct. 22, 2020 (CN) 202011137316.7
Oct. 18, 2021 (JP) 2021-170123

(51) Int. Cl. C12Q 1/70; C12Q 1/6823; C12Q 1/6848; B01L 7/00; B01L 3/00

(52) U.S. Cl. CPC C12Q 1/701; C12Q 1/6823; C12Q 1/6848; B01L 7/525; B01L 3/502; B01L 2300/0609; B01L 3/52; B01L 3/5023; B01L 2300/0681; B01L 2400/0677; B01L 3/567

(57) ABSTRACT

A nucleic acid detection system according to an embodiment is a nucleic acid detection system to detect a target nucleic acid in a sample and includes a thermal inactivation chamber, an amplification chamber, and a detection chamber, all of which constitute a liquid flow path. In this system, liquid flows through the thermal inactivation chamber, the amplification chamber, and the detection chamber sequentially. The thermal inactivation chamber includes a reagent to thermally inactivate and decompose the sample. The amplification chamber includes a reagent to amplify the target nucleic acid. The detection chamber includes a test strip to conduct a Cas enzyme reaction with the target nucleic acid and a lateral flow detection.

### CROSS-REFERENCE TO RELATED APPLICATIONS

This application is based upon and claims the benefit of priority from Chinese Patent Application No. 202011137316.7, filed on Oct. 22, 2020; and Japanese Patent Application No. 2021-170123, filed on Oct. 18, 2021, the entire contents of which are incorporated herein by reference.

### FIELD

The embodiments that are disclosed in this specification and drawings relate to a nucleic acid detection system and to a nucleic acid detection method.

### BACKGROUND

Conventional technologies to detect a nucleic acid is mainly based on the PCR detecting technology. The PCR detecting technology becomes a golden standard in the nucleic acid detection field. Specifically, as illustrated in FIG. 1, the flow of the nucleic acid detection includes processes such as sampling, nucleic acid extraction, amplification, detection, and result reporting. Not only this technology requires an expensive PCR instrument, a suitable polymerase, and a primer and/or a probe, but also a total operation time is long. This also requires not only a high professional degree to the operator but also a special experimental environment (approved PCR experimental room). In order to promptly obtain the detection result by simplifying the nucleic acid detection process, many attempts have been made by those skilled in the art. In U.S. Pat. No. 7,9680,406A, the system is described as the detection technology to integrate a plurality of the steps in which a plurality of target nucleic acids can be amplified in a single reaction compartment; with this system, the purpose of “sample-in-result-out” can be achieved. However, because this technology is still based on the PCR technology, not only the quasi-positive problem cannot be avoided, but also the detection instrument itself used is expensive. On top of these, a special experimental apparatus is necessary for detection, so that POCT (point-of-care testing) cannot be realized either.

The SHERLOCK technology (Specific High Sensitivity Enzymatic Reporter Unlocking) is the novel diagnose tool characterized by its promptness, high sensitivity, and low cost, which is developed with a novel system called CRISPR-Cas by Chinese American Feng Zhang; this can be used to detect the diseases such as Zika virus infection and dengue fever infection. This technology detects RNA in the sample by using a Cas13a protein having a “collateral shearing” activity and a special amplification technique to construct the detection system capable of precise detection of a single nucleic acid molecule in blood serum, urine, or saliva (see, Gootenberg, Jonathan S., et al. “Multiplexed and portable nucleic acid detection platform with Cas13, Cas12a, and Csm6”, Science 360, 6387 (2018): 439-444). However, all the conventional IVD (In Vitro Diagnosis) detection technologies by the SHERLOCK technology and other CRISPR require to separately carry out a plurality of steps such as sampling, nucleic acid extraction, amplification, Cas enzyme reaction, and detection, as illustrated in FIG. 1 or FIG. 2, so that during these processes there is a risk of pollution by operation such as opening of a cap. On top of this, there is a special requirement in an experimental environment, as well as, among other things, there are problems such as the need for storage and transporting environment in the liquid reaction system. Accordingly, there has been room for improvement in integration and automation.

### BRIEF DESCRIPTION OF THE DRAWINGS

FIG. 1 is a schematic drawing to compare the present embodiment with the nucleic acid detection technology in the conventional technology;

FIG. 2 is a schematic drawing illustrating the detection flow in the IVD detection technology by CRISPR;

FIG. 3 is a schematic drawing illustrating a first embodiment of the nucleic acid detection system according to the present embodiment;

FIG. 4 is a schematic drawing illustrating the specific composition of each chamber in the disposable detection part of the nucleic acid detection system according to the present embodiment;

FIG. 5 is a schematic drawing illustrating a step for sampling by using the nucleic acid detection system according to the present embodiment;

FIG. 6 is a schematic drawing illustrating the structure of the first chamber in the nucleic acid detection system according to the present embodiment;

FIG. 7 is a schematic drawing illustrating the structure of a wax valve in the nucleic acid detection system according to the present embodiment;

FIG. 8 is a schematic drawing illustrating the specific composition of the fourth chamber in the nucleic acid detection system according to the present embodiment;

FIG. 9 is a schematic drawing illustrating the reactions in each region at the time when a positive sample and a negative sample pass through the nucleic acid detection system according to the present embodiment;

FIGS. 10A and 10B are schematic drawings illustrating the coloring state of the test strip according to the present embodiment;

FIG. 11 is a schematic drawing illustrating a second embodiment of the nucleic acid detection system according to the present embodiment; and

FIG. 12 is a photograph of the test strips illustrating the detection results of Example 2.

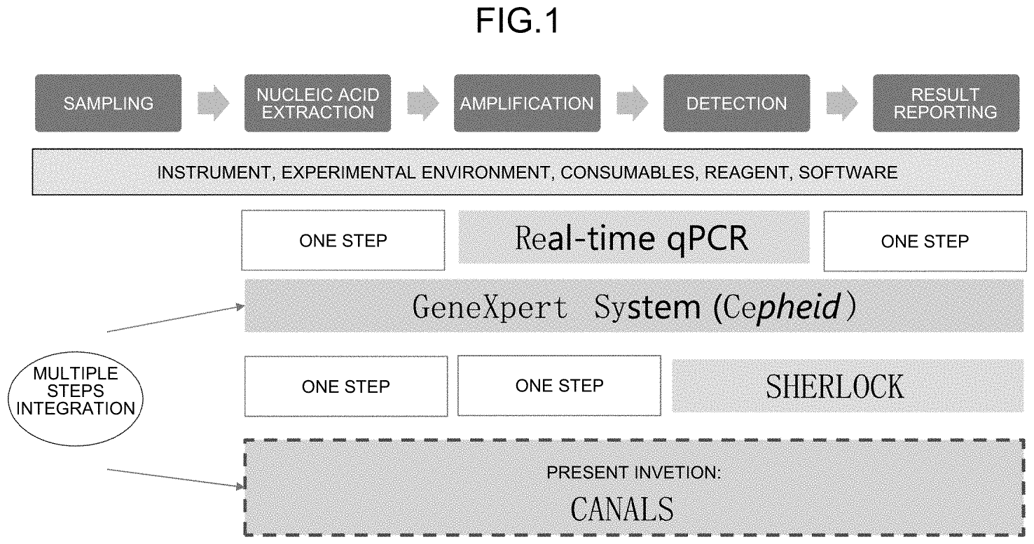

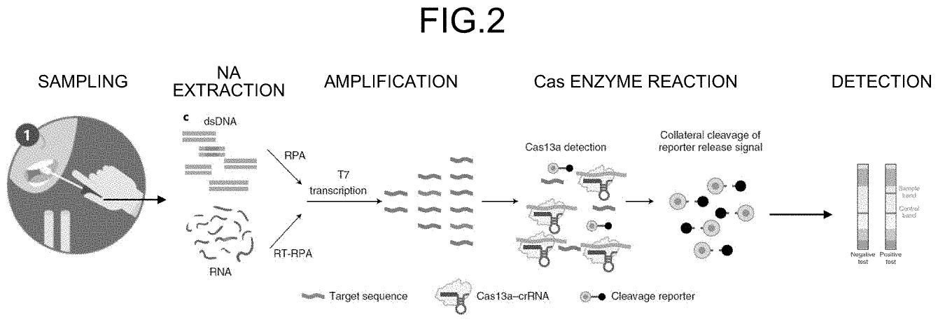

### DETAILED DESCRIPTION

One object of the embodiments disclosed in this specification and the drawings is to detect a nucleic acid with high sensitivity, although objects of the embodiments disclosed in this specification and the drawings is not limited to this object. Each effect on the corresponding composition illustrated in the embodiments to be described later may also be included as other objects.

A nucleic acid detection system according to an embodiment is a nucleic acid detection system to detect a target nucleic acid in a sample and includes a thermal inactivation chamber, an amplification chamber, and a detection chamber, all of which constitute a liquid flow path. In this system, liquid flows through the thermal inactivation chamber, the amplification chamber, and the detection chamber sequentially. The thermal inactivation chamber includes a reagent to thermally inactivate and decompose the sample. The amplification chamber includes a reagent to amplify the target nucleic acid. The detection chamber includes a test strip to conduct a Cas enzyme reaction with the target nucleic acid and a lateral flow detection.

Next, the present embodiment will be described in detail with referring to the drawings. The present embodiment is not limited to these embodiments, but includes all the embodiments satisfying the elements described in the claims of this application.

In view of the situation of the conventional technology as described above, the object of the present embodiment is to provide the nuclear acid detection system, among other things, that does not require expensive equipment, an expert operator, a special experimental room, and the like, that is capable of being stored and transported at room temperature, that can ensure a sequence specificity in the detection result, and that is portable, automated, and highly integrated, thereby not polluting inside and outside the system.

In the present embodiment, a target nuclear acid in a sample is detected by using a sequence specificity, in which the enzyme such as Cas12/13 is editable, and the collateral shearing activity; a continuous liquid flow path is formed by successively connecting specific first to fourth chambers; and a reagent composite body is rationally arranged, for example, on a paper-based material such that the nucleic acid detection result may be reported to a lateral flow test strip by a coloring reaction. With these, the automated portable nucleic acid detection system having a plurality of steps integrated could be provided, thereby realizing “sample-in-result-out”, application to POCT, and detection of the target nucleic acid in the sample; the integrated steps including sampling, nucleic acid extraction, amplification, Cas enzyme reaction, and detection coloring in the nucleic acid detection.

In addition, by simultaneously including a plurality of the fourth chambers in one system, the nucleic acid detection system of the present embodiment can realize simultaneous detection of a plurality of target nucleic acids, so that this system is applicable to, among other things, judgement of the gene type, for example, identification of the HPV gene type and MTB/RIF, and simultaneous detection of DNA and RNA.

According to the present embodiment, the nucleic acid detection system that is compact and has a high detection sensitivity can be provided.

### First Embodiment

A first embodiment according to the present embodiment relates to a nucleic acid detection system to detect a target nucleic acid in a sample, in which the system includes a first chamber to a fourth chamber that constitute a liquid flow path. In this system, liquid flows from the first chamber to the fourth chamber, in which the first chamber includes a swab inlet port to introduce a swab to which the sample is attached; the second chamber includes a reagent to thermally inactivate and decompose the sample; the third chamber includes a reagent to isothermally amplify the target nucleic acid; and the fourth chamber includes a test strip to conduct a Cas enzyme reaction with the target nucleic acid and a lateral flow detection. Here, the nucleic acid detection system has a wax valve arranged between the third chamber and the fourth chamber to partition these chambers, and has a temperature control unit to control the temperature of the third chamber and the wax valve.

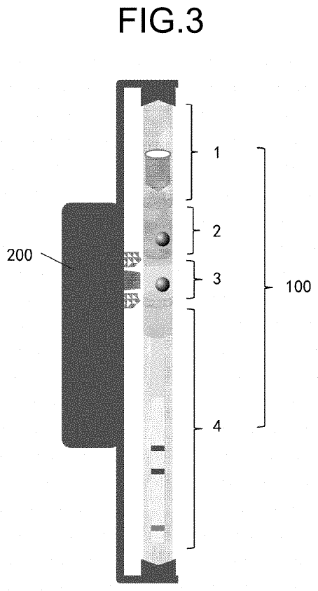

FIG. 3 is a schematic drawing illustrating the first embodiment of the nucleic acid detection system according to the present embodiment. As illustrated in FIG. 3, the nucleic acid detection system 10 includes a disposable detection part 100 and a temperature control unit 200. In the disposable detection part 100, the first chamber 1 to the fourth chamber 4 are successively connected from the upper side to the lower side of the drawing thereby forming a continuous liquid flow path. In the case when the disposable detection part is, for example, a paper-based material, the liquid can flow from the first chamber to the fourth chamber by a syphon effect or a capillary action.

There is no particular restriction in the paper-based material; so, various materials that are usually used in this field to prepare a disposable test strip may be used.

The disposable detection part 100 may be of a columnar shape or of a cuboid shape, such as a shape of a refill of a ballpoint pen. The length thereof is, for example, in the range of 5 to 15 cm, and the inner diameter thereof is, for example, in the range of 1 to 5 mm. The length and inner diameter thereof may be any within the above-described ranges. Therefore, the length may be 5 cm, 6 cm, 7 cm, 8 cm, 9 cm, 10 cm, 11 cm, 12 cm, 13 cm, 14 cm, 15 cm, and the like; the inner diameter may be 1 mm, 2 mm, 3 mm, 4 mm, 5 mm, and the like, indicating that they are not particularly restricted.

The temperature control unit 200 may also be disposable. For example, this may be made disposable by integrating with the disposable detection part 100. But a non-disposable type is preferable in view of a cost. The temperature control unit 200 may be of a shape like a hollow case with a nested type that matches to the disposable detection part 100, or of a glove-like shape with a semi-open type; so, for example, a hollow case shape such as a rod of a ballpoint pen may be mentioned. The temperature control unit 200 heats the chamber and the wax valve that correspond to the disposable detection part 100 at a prescribed temperature by a heating block H. When using, after the sampled swab is inserted into the disposable detection part 100, the whole or part of the disposable detection part 100 is inserted or buried into the temperature control unit 200; then, the temperature control unit 200 is started to carry out the prescribed temperature control program. Here, the temperature control unit is not essential; thus, in the case that the reaction undergoes at normal temperature, the nucleic acid detection system according to the present embodiment may not include the temperature control unit.

### First Chamber

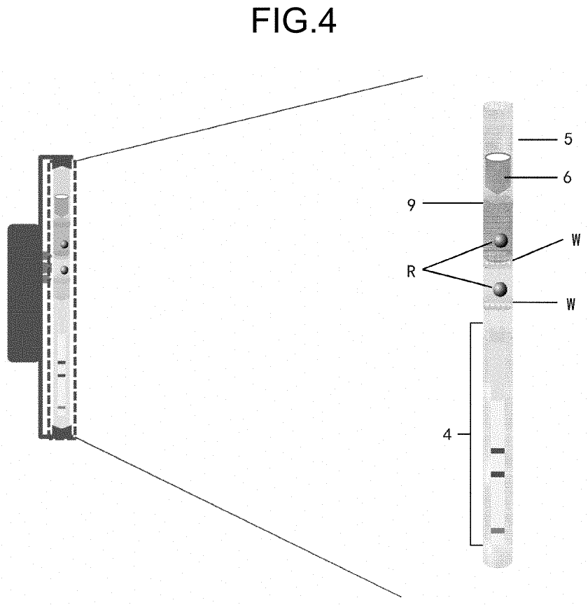

FIG. 4 is a schematic drawing illustrating the specific composition of each chamber in the disposable detection part of the nucleic acid detection system that is illustrated in FIG. 3. As illustrated in FIG. 4, the first chamber 1 includes a swab inlet port 5 into which the swab to which the sample is attached is inserted.

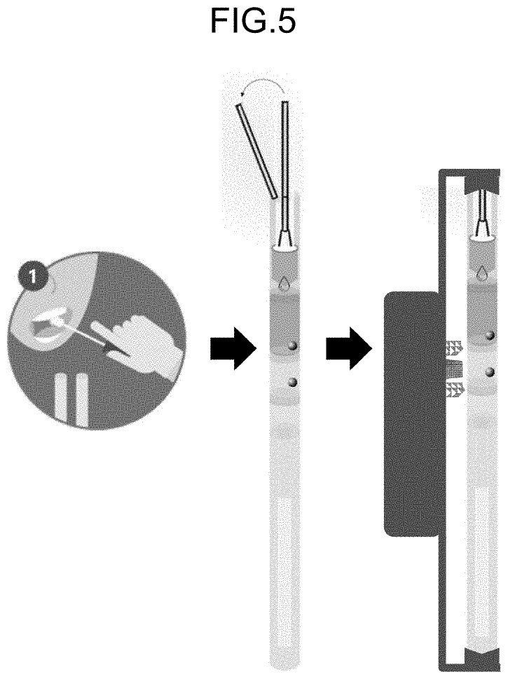

FIG. 5 is a schematic drawing illustrating a step for sampling by using the nucleic acid detection system according to the present embodiment. As illustrated in FIG. 5, after sampling by using a swab, the swab is inserted into the swab inlet port 5 of the nucleic acid detection system; then, the swab is pressed to a wall of the system, or by flowing a liquid sample including a target nucleic acid into a liquid flow path by a pushing-out path 6, then the swab is folded. Next, the disposable detection part 100 is inserted or buried into the temperature control unit 200; then, the liquid sample having entered into the liquid flow path flows from the first chamber 1 to the fourth chamber 4 by the syphon effect or by the capillary action.

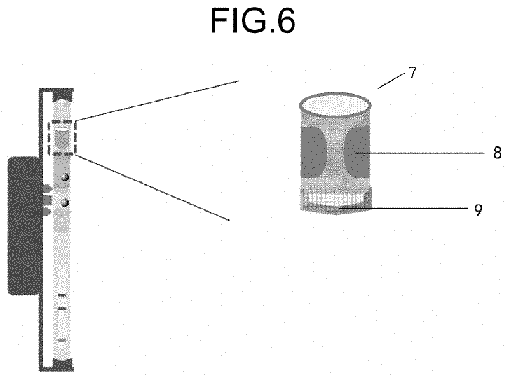

FIG. 6 is a schematic drawing illustrating the structure of the first chamber in the nucleic acid detection system according to the present embodiment. The structure of the first chamber is not particularly restricted so far as the swab can be inserted. For example, this may be the one having a composition like a pushing-out funnel 7; specifically, this may be funnel-like having a pressing protrusion 8 in the inner wall thereof, as illustrated in FIG. 6. It is preferable to further arrange a filter 9 between the first chamber 1 and the second chamber 2 to purify the sample by rough filtration.

Note that the first chamber is not necessarily provided to the nucleic acid detection system. For example, when the second chamber is provided with a cap that has the pushing-out funnel 7, the nucleic acid detection system may not be provided with the first chamber. The first chamber is also called a sample chamber.

### Second Chamber

As illustrated in FIG. 4, the second chamber 2 includes a reagent to thermally inactivate and decompose the sample. After entering into the second chamber 2 after passing through the filter 9, the liquid sample is hydration-activated, then thermally inactivated and decomposed by the reagent included therein. The reagent used for the thermal inactivation and decomposition is, for example, a buffer solution including an enzyme that is used for inactivation and decomposition.

Illustrative examples of the enzyme include a protease K, an RNase nuclease and/or a peptidase. By action of these enzymes, a membrane protein, a protein that is bonded to DNA and RNA, and the like can be decomposed, resulting in dissolution of virusware to release nucleic acids such as DNA and RNA into the sample. Here, the enzyme may undergo the reaction at normal temperature; but the reaction may be carried out at high temperature by selecting the enzyme species that has a high-temperature resistance. In this case, by heating this chamber, the target nucleic acid in the sample can be further inactivated.

In addition to the enzyme, the buffering agent may include, without particular restriction, a general ingredient such as EDTA or Tris-HCl that is included when the buffer solution is prepared in this field.

The reagent included in the second chamber 2 in the nucleic acid detection system according to the present embodiment is preferably in the form of a freeze-dried reagent ball R, or a freeze-dried reagent that is adsorbed to a holder material P (the same is applied to the reagents described hereinafter).

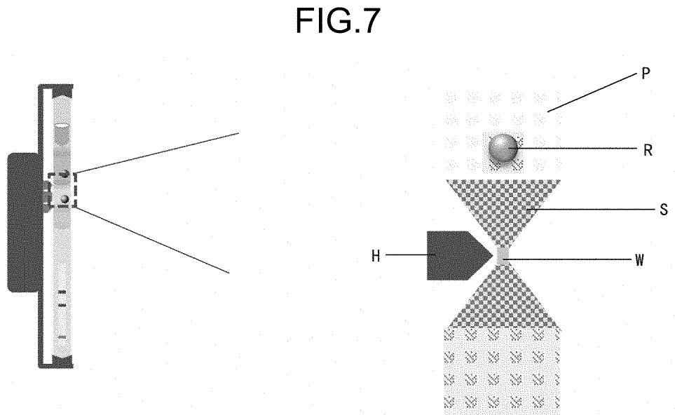

FIG. 7 is a schematic drawing illustrating the structure of the wax valve in the nucleic acid detection system according to the present embodiment. As illustrated in FIG. 7, between the second chamber 2 and the third chamber 3, a thermally unstable valve, for example, a wax valve W may be arranged to partition these chambers. The wax valve may be formed by a single wax layer or may be adsorbed to the holder material P. An absorbing material S may be formed around the wax valve W; so, the wax valve is absorbed into the absorbing material S after melted. The wax valve W is opened during heating (the same is applied to the wax valve W between the third chamber and the fourth chamber—this will be described later) to cause the liquid sample to flow from the second chamber to the third chamber.

In the present embodiment, the case is described when the wax valve is used, but the valve is not limited to this; so, any heretofore known valve may be used. Illustrative examples of the heretofore known valve include a valve that is melted and opened by an electric stimulation and an electrically operated valve. Here, the second chamber is also called a thermal inactivation chamber.

### Third Chamber

As illustrated in FIG. 4, the third chamber 3 includes the reagent to amplify the target nucleic acid. With this, the liquid sample is amplified, for example, isothermally amplified in this chamber. The isothermal amplification may be any one selected from an amplification by a nucleic acid sequence, an amplification by a recombinant polymerase enzyme, a loop-mediated isothermal amplification, a chain-substitution amplification, a ribonuclease-dependent amplification, and a slit enzyme amplification. To facilitate amplification, the temperature of the third chamber 3 may be controlled and/or the wax valve W may be further heated by using the temperature control unit 200. With this, the flow of the liquid sample between the chambers can be controlled.

The reagent and method that are used in amplification may be configured by referring to the descriptions with regard to the conventional technologies. There is no particular restriction in this; for example, those described in the following documents may be used: Tsugunori Notomi et al. “Loop-mediated isothermal amplification of DNA” Nucleic Acids Res. 2000 Jun. 15; 28 (12): e63 or U.S. Pat. No. 7,270,981). Here, the third chamber is also called the amplification chamber.

### Fourth Chamber

When the wax valve W between the third chamber 3 and the fourth chamber 4 is heated thereby being opened by melting, the liquid sample flows into the fourth chamber 4.

As illustrated in FIG. 4, the fourth chamber 4 includes the test strip to carry out the Cas enzyme reaction with the target nucleic acid in the sample and the lateral flow detection.

The reagent to carry out the Cas enzyme reaction includes a Cas enzyme complex. Specifically, the Cas enzyme complex may include the Cas enzyme, a guide nucleic acid, and a probe. The guide nucleic acid includes a guide sequence capable of bonding with the target nucleic acid in the sample, and can form a complex with the Cas enzyme. The probe may also be a molecule based on DNA or RNA that includes a non-target nucleic acid sequence. When this is sheared by the Cas enzyme, a molecule that is necessary for the subsequent reaction can be generated.

Illustrative examples of the Cas enzyme include Cas12, Cas13, and Cas14. They may have a shearing activity to the target nucleic acid, and this is at least one selected from, for example, Cas12a, Cas13a, Cas13b, Cas14a, Cas14b, and Cas14c, and these may have a shearing activity to the target nucleic acid, and this is at least one selected from, for example, dCas12a, dCas13a, dCas13b, dCas14a, dCas14b, and dCas14c. For example, when the detection by the nucleic acid detection system according to the present embodiment is only as to whether or not the target nucleic acid is present, the Cas enzyme is not necessary to be active. However, this may be active. Here, the fourth chamber is also called the detection chamber.

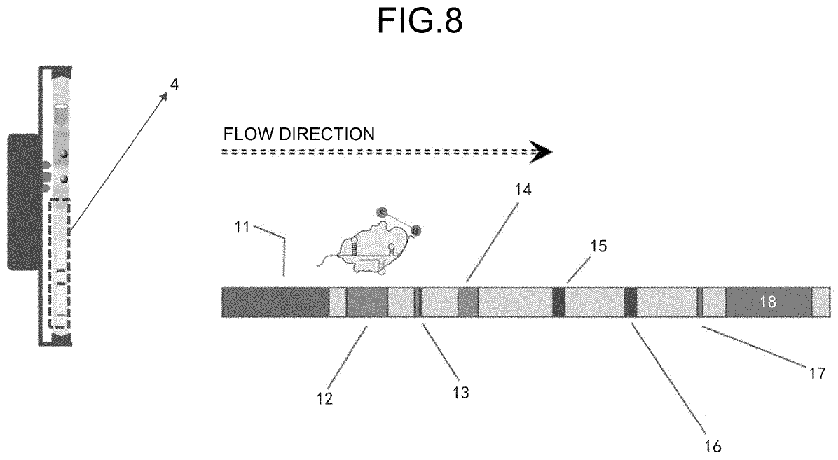

FIG. 8 is a schematic drawing illustrating the specific composition of the fourth chamber in the nucleic acid detection system according to the present embodiment.

From left to right, the chamber is composed of a sample pad 11, a Cas enzyme reaction region 12 (Cas enzyme complex), a Cas enzyme reaction termination region 13 (Cas enzyme antibody or inhibitor), a gold-labelled anti-FAM antibody region 14, a streptavidin region 15 (reference band 1), an antibody capturing region 16 (test band), an anhydrous copper sulfate band 17 (reference band 2), and an absorption pad 18.

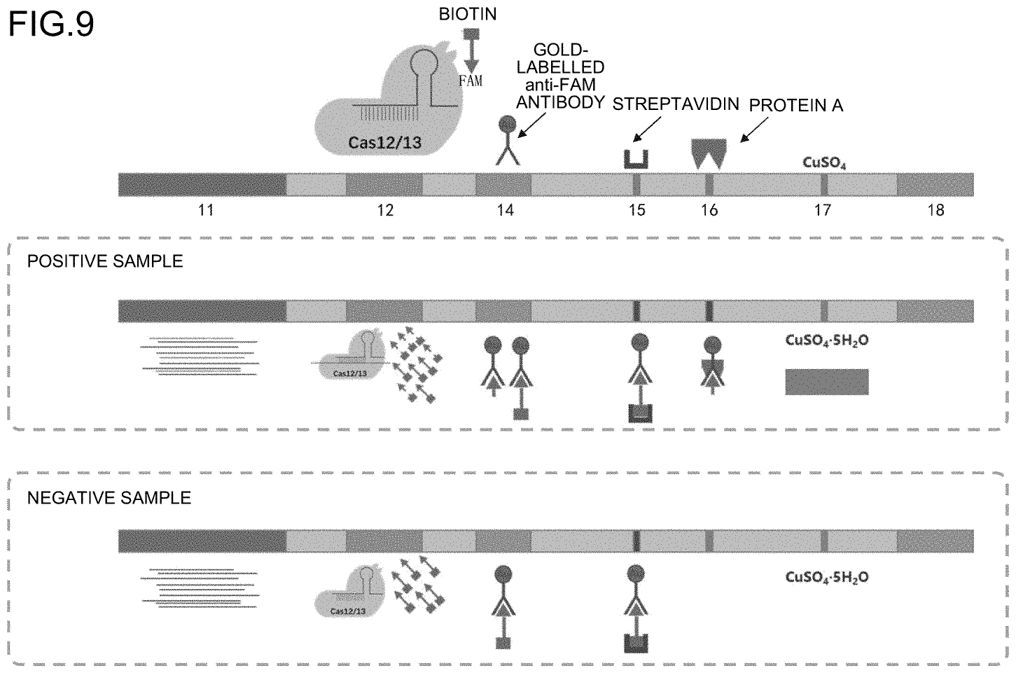

FIG. 9 is a schematic drawing illustrating the reactions in each region at the time when a positive sample and a negative sample pass through the nucleic acid detection system according to the present embodiment.

Next, with referring to FIG. 8 and FIG. 9, detection and coloring state of the positive and negative samples in the following Step S11 to Step S16 in the fourth chamber 4 will be described.

Step S11: After the liquid sample (hereinafter, this is also called simply “liquid”) enters into the sample pad 11, firstly this passes through the Cas enzyme reaction region, where the positive sample activates the Cas enzyme complex to cleave the probe (reporting sequence) thereby liberating FAM and biotin. On the other hand, the probe is not cleaved in the negative sample.

Step S12: After the products of the Cas enzyme reaction pass through the Cas enzyme reaction termination section, the Cas enzyme reaction is completed. The Cas enzyme is captured, so that the forward flow thereof cannot continue any more. With this, the reaction efficiency and time of the detection can be ensured.

Step S13: The forward flow of the liquid continued; then, the cleaved and uncleaved reporting sequences bonded with an anti-FITC and a FAM antibody in the gold-labelled anti-FAM antibody region.

Step S14: When the liquid continued to flow and reached the streptavidin region, biotin was captured and the gold-labelled particles agglomerated to generate a color (reference band 1). In this region, the color is generated regardless whether or not the reporting sequence is cleaved.

Step S15: When the liquid continued to flow and reached the antibody capturing region, the FAM terminal of the cleaved reporting sequence+FITC and FAM antibody+gold-labelled particles bonded to the antibody capturing region; then, the gold-labelled particles were agglomerated to generate a color (namely two-line coloring). In the case of the negative sample, the reporting sequence is not cleaved, and whole of the reporting sequence has been bonded to the streptavidin region, so that the antibody capturing region does not generate a color.

Step S16: Next, the liquid passed through the anhydrous copper sulfate band, then reached the absorption pad. Here, regardless of whether or not the target sequence is present in the sample, when the aqueous solution flows through the anhydrous copper sulfate band, a blue color is generated. The blue color indicates that the amount of the liquid was sufficient, thereby indicating that the liquid flows the test strip thoroughly. On the other hand, when the anhydrous copper sulfate band (reference band 2) does not generate a color, this is judged that the detection is invalid. With this, quality of the validity of the detection of the entire test strip can be controlled.

As can be clearly seen above, it can be judged that the detection result is valid only when both the reference band 1 and the reference band 2 simultaneously generate the colors. By arranging, in addition to the reference band 1, the anhydrous copper sulfate band (reference band 2) in such a way as the present embodiment, the quasi-negative problem (invalid detection) due to the insufficient sample amount in the conventional technology, in which the test strip is designed such that the test band is arranged after the reference band, can be solved. Therefore, according to the present embodiment, it can be seen that the accuracy of the nucleic acid detection can be improved as well.

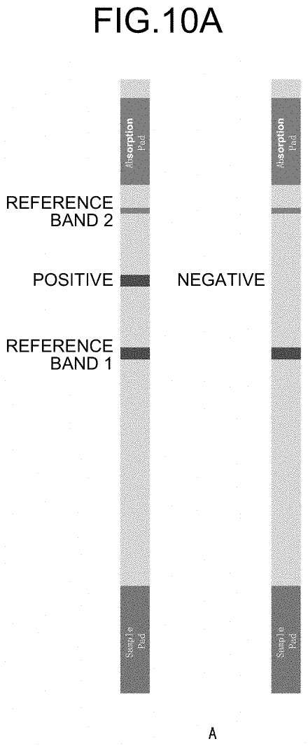

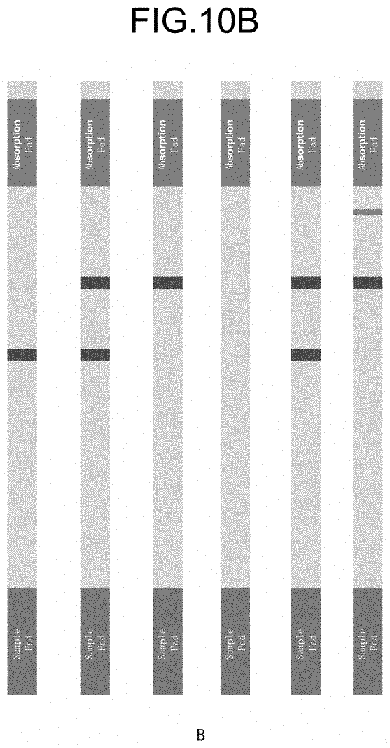

FIG. 10A and FIG. 10B are schematic drawings illustrating the coloring state of the test strip according to the present embodiment. FIG. 10A is the schematic drawing illustrating the coloring states that are judged to be valid when the positive sample and the negative sample pass through the nucleic acid detection system according to the present embodiment. FIG. 10B is the schematic drawing illustrating various coloring states that are judged to be invalid.

As described above, the nucleic acid detection system according to the first embodiment is the nucleic acid detection system to detect the target nucleic acid in the sample, in which the system includes the thermal inactivation chamber, the amplification chamber, and the detection chamber, all of which constitute the liquid flow path. The liquid flows the thermal inactivation chamber, the amplification chamber, and the detection chamber sequentially. The thermal deactivation chamber includes the reagent to thermally inactivate and decompose the sample. The amplification chamber includes the reagent to amplify the target nucleic acid. The detection chamber includes the test strip to carry out the Cas enzyme reaction with the target nucleic acid and the lateral flow detection. By the nucleic acid detection system according to the first embodiment as described above, the nucleic acid can be detected with a high sensitivity.

Here, it is preferable that the nucleic acid detection system is further provided, before the thermal inactivation chamber, with a sample chamber that includes a swab inlet port into which the swab to which a sample is attached can be inserted.

### Second Embodiment

The second embodiment according to the present embodiment is the nucleic acid detection system to simultaneously detect a plurality of target nucleic acids in a sample, in which the system includes the nucleic acid detection system according to the first embodiment, and the fourth chamber includes two or more test strips.

With the same principle and action of the nucleic acid detection system according to the first embodiment, the liquid flows from the first chamber 1 to the fourth chamber 4. In the present embodiment, however, the fourth chamber is branched to a plurality of channels so that a plurality of target nucleic acids may be detected in parallel. The editable Cas complex in each channel may be different so that different target nucleic acids can be detected.

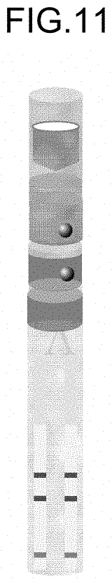

FIG. 11 is a schematic drawing illustrating the second embodiment of the nucleic acid detection system according to the present embodiment. This nucleic acid detection system has two fourth chambers (lateral flow test strips) so that simultaneous detection of two target nucleic acids can be realized.

The detection system of the second embodiment according to the present embodiment can also be applied to judgement of the gene type, for example, identification of the HPV gene type and MTB/RIF, and simultaneous detection of DNA and RNA. For example, when each Cas complex included in two or more test strips is designed for respective different target sequences, the gene type of the target nucleic acid in the sample can be judged.

Therefore, the present embodiment is the method to judge the gene type of the target nucleic acid in a sample, which uses the nucleic acid detection system described in the second embodiment. In the present embodiment, the Cas complexes included in the two or more test strips are designed for respective different target sequences.

Also, the present embodiment relates to the method to detect the target nucleic acid in a sample, in which the nucleic acid detection system described in the first or the second embodiment is used, and the method includes sampling, the process at which the swab thereof is inserted into the swab inlet port of the detection system, the process at which the temperature control unit is set to ON, and the process at which the color change of the test strip is observed in the fourth chamber. Illustrative examples of the sample include urine, blood, blood serum, cerebrospinal fluid, and saliva.

### EXAMPLES

Hereinafter, the nucleic acid detection system and the nucleic acid detection method according to the present embodiment are specifically described, taking a novel coronavirus (hereinafter, this is also described “severe acute respiratory syndrome coronavirus 2 (SARS-CoV-2)”) as the example, although the present embodiment is not limited to these Examples. Here, the novel coronavirus infection is also described “COVID-19”.

### Example 1: Preparation of Nucleic Acid Detection System

From the novel coronavirus genome sequence, the LAMP primer for the LAMP isothermal amplification of the N gene thereof and the crRNA sequence that detects this by using the Cas12 enzyme were designed.

The template is the plasmid that includes the N gene fragment of SARS-CoV-2, in which the N gene is as follows (Sequence No. 1)

Sequence No. 1:
CCGAAGAGCTACCAGACGAATTCGTGGTGGTGACGGTAAAATGAAAGATC
TCAGTCCAAGATGGTATTTCTACTACCTAGGAACTGGGCCAGAAGCTGGA
CTTCCCTATGGTGCTAACAAAGACGGCATCATATGGGTTGCAACTGAGGG
AGCCTTGAATACACCAAAAGATCACATTGGCACCCGCAATCCTGCTAACA
ATGCTGCAATCGTGCTACAACTTCCTCAAGGAACAACATTGCCAAAAGGC
TTCTACGCAGAAGGGAGCAGAGGCGGCAGTCAAGCCTCTTCTCGTTCCTC
ATCACGTAGTCGCAACAGTTCAAGAAATTCAACTCCAGGCAGCAGTAGGG
GAACTTCTCCTGCTAGAATGGCTGGCAATGGCGGTGATGCTGCTCTTGCT
TTGCTGCTGCTTGACAGATTGAACCAGCTTGAGAGCAAAATGTCTGGTAA
AGGCCAACAACAACAAGGCCAAACTGTCACTAAGAAATCTGCTGCTGAGG
CTTCTAAGAAGCCTCGGCAAAAACGTACTGCCACTAAAGCATACAATGTA
ACACAAGCTTTCGGCAGACGTGGTCCAGAACAAACCCAAGGAAATTTTGG
GGACCAGGAACTAATCAGACAAGGAACTGATTACAAACATTGGCCGCAAA
TTGCACAATTTGCCCCCAGCGCTTCAGCGTTCTTCGGAATGTCGCGCATT
GGCATGGAAGTCACACCTTCGGGAACGTGGTTGACCTACACAGGTGCCAT
CAAATTGGATGACAAAGATCCAAATTTCAAAGATCAAGTCATTTTGCTGA
ATAAGCATATTGACGCATACAAAACATTCCCACCAACAGAGCCTAAAAAG
GACAAAAAGAAGAAGGCTGATGAAACTCAAGCCTTACCGCAGAGACAGAA
GAAACAGCAAACTGTGACTCTTCTTCCTGCTGCAGATTTGGATGATTTCT
CCAAACAATTGCAACAATCCATGAGCAGTGCTGACTCAACTCAGGCCTAA

In Table 1 below, the LAMP primer sequences used in Examples are described.

TABLE 1

| | |
|---|---|
| N-gene F3-2 | GCTGCAATCGTGCTACAACT |
| (Sequence No. 2) | |
| N-gene B3-2 | TCTGTCAAGCAGCAGCAAAG |
| (Sequence No. 3) | |
| N-gene FIP-2 | TGCGACTACGTGATGAGGAACGTTGCCAAAA |
| (Sequence No. 4) | GGCTTCTACGC |
| N-gene BIP-2 | TTCAACTCCAGGCAGCAGTAGGCAAGAGCAG |
| (Sequence No. 5) | CATCACCGC |
| N-gene LF-2 | TTGACTGCCGCCTCTGC |
| (Sequence No. 6) | |
| N-gene LB-2 | GGAACTTCTCCTGCTAGAATGGC |
| (Sequence No. 7) | |

The designed crRNA sequence (sequence No. 8) is as follows.

Sequence No. 8:
UAAUUUCUACUAAGUGUAGAUUUGAACUGUUGCGACUACGU

Probe sequence: FITC-T12-Biotin

All the materials described above are synthesized by and purchased from Nanjing Genscript Biotech Corp.

The LAMP amplification may be conducted with referring to the specification of the WarmStart (registered tradename) LAMP kit.

The test paper for detection of SARS-CoV-2 was prepared by the following Steps S21 to S29.

Step S21: Glass fiber filter (Whatman, 1827-021) was cut to a prescribed size.

Step S22: High pressure sterilization is conducted for 90 minutes (high pressure sterilizer, MKII).

Step S23: Blocking is conducted for 12 hours in the nuclease-free 5% BSA (EM Millipore, 126609-10GM).

Step S24: Cleaning is conducted three times with nuclease-free water (Life Technologies, AM9932).

Step S25: After 4% RNASecure (Life Technologies, AM7006) is added, this is allowed to stand at 60° C. for 20 minutes; then, cleaning is conducted three times with nuclease-free water.

Step S26: The treated paper is dried at 80° C. in an oven (DHG-9140A, Shanghai Yiheng) for 15 minutes.

Step S27: Components in the respective chambers are arranged in the test paper that is treated as described above in accordance with design. Specifically, they are (a) to (d) as described below.

(a) Second chamber

TABLE 2

| | Volume |
|---|---|
| Reagent | (μL) |
| 50 mM TCEP (ab142040, Abcam) | 1 |
| 2 mM EDTA (T9191, TaKaRa) | 1 |
| RNase inhibitor (M0314S, NEB) | 1 |
| 10 mM Tris-HCl (pH 8.0) | 10 |
| 500 mM Trehalose (TS1M-100, Life Sciences | 1 |
| Advanced Technologies) | |
| Achromopeptidase (ACP, A3547, Sigma Aldrich) | 5 |
| H₂O | 1 |
| Total volume | 20 |

(b) Third chamber: Mixed solution for amplification is described in Table 3.

TABLE 3

| | Volume |
|---|---|
| Reagent | (μL) |
| WarmStart LAMP 2× Master Mix (E1700S, NEB) | 12.5 |
| Primer Mix (LAMP primer described in Table 1) | 2.5 |
| H₂O | 10 |
| Total volume | 25 |

(c) Here, carnauba wax (C804522, MACKLIN) is used as the wax valve.

(d) Fourth chamber: The reagents and other ingredients used in the Cas enzyme reaction region are described in Table 4 and Table 5.

TABLE 4

| | Final | Volume |
|---|---|---|
| Reagent | concentration | (μL) |
| LbaCas12a (M0653S, NEB) | 1 μM | 1 |
| crRNA (Sequence No. 8) | 1 μM | 1 |
| Probe | 10 μM | 1 |
| H₂O | | 17 |
| Total volume | | 20 |

TABLE 5

| | Volume |
|---|---|
| Other ingredient | (μL) |
| Cas enzyme inhibitor, AcrVA (Genscript) | 1 |
| Streptavidin (N7021S, NEB) | 1 |
| Gold-labelled anti-FAM antibody | 1 |
| (ab19491, abcam) | |
| Protein A (ab84187, abcam) | 1 |
| Copper sulfate (C805358, MACKLIN) | 1 |

Step S28: Rapid cooling by liquid nitrogen is conducted, and then, one overnight freeze drying is conducted.

Step S29: The test paper prepared under a dry environment is fabricated to a sleeve material.

### Example 2: Detection of SARS-CoV-2 by the Nucleic Acid Detection System of Example 1

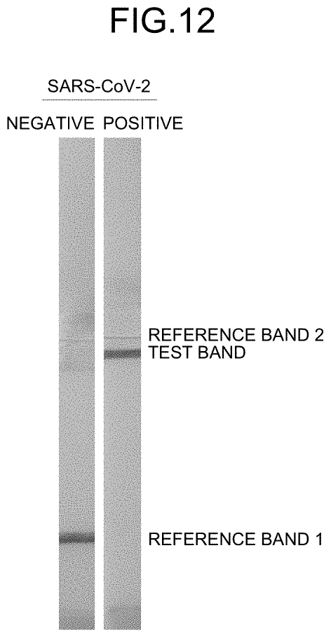

The detection test paper for SARS-CoV-2 in Example 1 was combined with the temperature control unit; then, a positive plasmid solution (positive sample) and a solution not containing SARS-CoV-2 (negative sample), volume of 50 μL each, were dropped into the first chamber. The temperature control unit was operated in accordance with a prescribed program to cause the sample to flow from the first chamber into the second chamber. In this Example, a simulated sample was used, so that the thermal inactivation and decomposition in the second chamber were not necessary; so, the sample flowed to the third chamber as it was. Temperature of the third chamber was controlled at 65° C. to carry out the LAMP amplification in this chamber; then, 10 minutes thereafter, the wax valve was opened to cause the liquid to flow into the fourth chamber. The temperature of the fourth chamber was controlled at 37° C. In this chamber, first, the liquid enters into the Cas enzyme reaction region to hydrate the freeze-dried reagents in this region; then, the previously designed Cas enzyme recognizes the target nucleic acid to shear the probe. The reaction time is 5 minutes. The sheared probe continued to move toward the water-absorption pad in the test strip. In accordance with the designed principle, firstly the reference band 1 was colored to red, then the test band was colored to red, and finally the anhydrous copper sulfate band (reference band 2) was colored to blue. In the positive sample, all these three bands are colored; on the contrary, in the negative sample, the test band is not colored. The photo of the test strip (fourth chamber) illustrating the detection result is described in FIG. 12.

As can be seen above, according to the present embodiment, the nuclear acid detection system can be provided that does not require expensive equipment, an expert operator, and a special experimental room, that can be stored and transported at room temperature, that can ensure a sequence specificity in the detection result, that does not pollute inside and outside the system, that can realize “sample-in-result-out”, and that is applicable to POCT, and is portable, automated, and highly integrated. When having a plurality of the fourth chambers, the nucleic acid detection system according to the present embodiment can simultaneously detect a plurality of target nucleic acids, and can be applied, for example, to judgement of the gene type, to simultaneous detection of DNA and RNA, and the like.

According to at least one embodiment described above, a nucleic acid can be detected with a high sensitivity.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A nucleic acid detection system to detect a target nucleic acid in a sample, the nucleic acid detection system comprising a thermal inactivation chamber, an amplification chamber, and a detection chamber, all of which constitute a liquid flow path;
liquid flows through the thermal inactivation chamber, the amplification chamber, and the detection chamber sequentially;
the thermal inactivation chamber includes a reagent to thermally inactivate and decompose the sample;
the amplification chamber includes a reagent to amplify the target nucleic acid; and
the detection chamber includes a test strip to conduct a Cas enzyme reaction with the target nucleic acid and a lateral flow detection.

2. The nucleic acid detection system according to claim 1, further comprising, before the thermal inactivation chamber, a sample chamber that includes a swab inlet port into which a swab to which the sample is attached is inserted.

3. The nucleic acid detection system according to claim 2, further comprising, between the sample chamber and the thermal inactivation chamber, a filter to purify the sample.

4. The nucleic acid detection system according to claim 2, wherein the sample chamber further comprises a pushing-out path.

5. The nucleic acid detection system according to claim 1, further comprising, between the thermal inactivation chamber and the amplification chamber, a valve to partition these chambers.

6. The nucleic acid detection system according to claim 1, further comprising, between the amplification chamber and the detection chamber, a valve to partition these chambers.

7. The nucleic acid detection system according to claim 5, wherein the valve is a wax valve.

8. The nucleic acid detection system according to claim 7, wherein the wax valve is composed of an independent wax layer or is adsorbed to a holder material.

9. The nucleic acid detection system according to claim 1, further comprising a temperature control unit to control a temperature of the amplification chamber.

10. The nucleic acid detection system according to claim 7, wherein the wax valve is opened by heating with a temperature control unit.

11. The nucleic acid detection system according to claim 1, wherein the reagents in the thermal inactivation chamber and the amplification chamber are freeze-dried agents.

12. The nucleic acid detection system according to claim 11, wherein the freeze-dried agents are a freeze-dried reagent ball or adsorbed to a holder material.

13. The nucleic acid detection system according to claim 1, wherein
the reagent that is used in the Cas enzyme reaction comprises a Cas enzyme complex,
the Cas enzyme complex comprises a Cas enzyme, a guide nucleic acid, and a probe,
the guide nucleic acid comprises a guide sequence capable of bonding to the target nucleic acid and is capable of forming a complex with the Cas enzyme, and
the probe is a molecule based on DNA or RNA comprising a non-target nucleic acid sequence and generates, when sheared by the Cas enzyme, a molecule that is necessary for a subsequent reaction.

14. The nucleic acid detection system according to claim 1, wherein the reagent to carry out the Cas enzyme reaction is a freeze-dried agent.

15. The nucleic acid detection system according to claim 14, wherein the freeze-dried agent is a freeze-dried reagent ball or adsorbed to a holder material.

16. The nucleic acid detection system according to claim 1, wherein the amplification is any one selected from an amplification by a nucleic acid sequence, an amplification by a recombinant enzyme polymerase, a loop-mediated isothermal amplification, a chain-substitution amplification, a ribonuclease-dependent amplification, and an amplification by a slit enzyme.

17. The nucleic acid detection system according to claim 1, wherein
the detection chamber comprises two or more test strips, and
by using the two or more test strips, a plurality of target nucleic acids in the sample are simultaneously detected.

18. A nucleic acid detection method to detect a target nucleic acid in a sample, the nucleic acid detection method comprising:
by using a nucleic acid detecting system that comprises a thermal inactivation chamber, an amplification chamber, and a detection chamber, all of which constitute a liquid flow path, in which liquid flows through the thermal inactivation chamber, the amplification chamber, and the detection chamber sequentially,
thermally inactivating and decomposing the sample in the thermal inactivation chamber;
amplifying the target nucleic acid in the amplification chamber; and
conducting a Cas enzyme reaction with the target nucleic acid and a lateral flow detection in the detection chamber.

19. The nucleic acid detection method according to claim 18, wherein
the nucleic acid detection system further comprises, before the thermal inactivation chamber, a sample chamber that includes a swab inlet port into which a swab to which the sample is attached is inserted, and
the swab is inserted into the swab inlet port in the sample chamber.

20. The nucleic acid detection method according to claim 18, wherein in the detection chamber, a change of a color in a test strip is observed.

21. The nucleic acid detection method according to claim 18, wherein the sample is urine, blood, blood serum, cerebrospinal fluid, or saliva.

22. The nucleic acid detection method according to claim 18, wherein
the detection chamber comprises two or more test strips,
each Cas complex included in the two or more test strips is designed for respective different target sequences, and
by using the two or more test strips, a plurality of target nucleic acids in the sample are simultaneously detected.
